(19) United States
(12) Patent Application Publication
(10) Pub. No.: US 2024/0358048 A1
(43) Pub. Date: Oct. 31, 2024

(54) SOLID FILAMENTOUS FUNGUS CULTURE, COMPOSITION, FOOD, ANIMAL FEEDSTUFF, FOOD ADDITIVE, OR ANIMAL FEEDSTUFF ADDITIVE, AND METHOD FOR PRODUCING SOLID FILAMENTOUS FUNGUS CULTURE

(71) Applicants: Fujiwara Techno-Art Co., Ltd., Okayama (JP); MITSUI & CO., LTD., Tokyo (JP)

(72) Inventors: Satoko Senoo, Okayama (JP); Natsuki Fukano, Okayama (JP); Akira Mori, Okayama (JP); Masahiro Kariyama, Okayama (JP); Naoki Oshida, Tokyo (JP); Jun Kamiya, Tokyo (JP)

(21) Appl. No.: 18/682,660

(22) PCT Filed: Jul. 15, 2022
(86) PCT No.: PCT/JP2022/027888

§ 371 Date: Feb. 9, 2024

(30) Foreign Application Priority Data
Aug. 13, 2021 (JP) 2021-132039

(51) Int. Cl. A23L 7/104; A23K 10/12; A23K 10/30; A23L 7/10; C12N 1/14

(52) U.S. Cl. CPC A23L 7/104; A23K 10/12; A23K 10/30; A23L 7/198; C12N 1/14

(57) ABSTRACT

Provided is a corn wet milling-derived article having a reduced sulfur dioxide concentration. The corn wet milling-derived article is a solid filamentous fungus culture. The corn wet milling-derived article may be one or more selected from the group consisting of corn gluten meal, corn gluten feed, and corn germ. The sulfur dioxide content in the solid filamentous fungus culture may be 30 ppm or less. The filamentous fungus may be non-mycotoxin-producing microbes. The solid filamentous fungus culture may include an enzyme produced by the filamentous fungus. The solid filamentous fungus culture may include polysaccharides that constitute the fungus body of the filamentous fungus.

### DESCRIPTION

### Technical Field

The present invention relates to a solid filamentous fungus culture.

### Background Art

Wet milling is one of the methods for industrial production of corn starch from corn. A wet milling process includes the five basic steps for separating corn into its four components starch, oil (germ oil), fiber, and protein: (1) careful selection of corn kernels; (2) soaking of corn kernels; (3) germ separation; (4) griding and fiber separation; and (5) separation of starch and gluten. These steps produce corn wet milling products including by-products such as corn gluten meal, corn germ, and corn gluten feed.

In the process, the soaking step (2) uses an approximately 0.1 to 0.3% sulfurous acid solution to soften the corn kernels and to make components easy to separate. While the sulfurous acid can be removed by final washing in the production of corn starch, a relatively large amount of the sulfurous acid may remain in corn gluten meal and other corn wet milling products, which is a problem due to the difficulty in removing the sulfurous acid even by washing.

While sulfurous acid is used for some purposes, such as prevention of oxidation and discoloration of foods, the use of sulfurous acid in foods is restricted in Japan such that the amount of sulfur dioxide remaining in foods must be less than 30 ppm, except for specially designated items. At present, therefore, corn gluten meal and other corn wet milling products are mainly used as livestock feed. However, they can also be used for foods if the remaining amount of sulfur dioxide can be reduced to less than the specified amount.

To address this problem, for example, Patent Document 1 proposes a method including heating corn gluten meal powder at a temperature of 105°° C. to 150°° C. to reduce its sulfur dioxide content to less than 30 ppm.

Patent Document 2 proposes another method including treating a corn protein product, such as corn gluten meal, with an oxidant (preferably hydrogen peroxide) to reduce its free sulfite concentration to less than 150 ppm.

### CITATION LIST

### Patent Document

Patent Document 1: Japanese Unexamined Patent Application, Publication No. 2011-97928

Patent Document 2: PCT International Publication No. WO2017/165748

### DISCLOSURE OF THE INVENTION

### Problems to be Solved by the Invention

Unfortunately, the method including heating to reduce the sulfur dioxide content may cause protein denaturation, alteration, or discoloration and has a problem in that the heating conditions, such as the heating temperature and time, must be controlled depending on the content of water in the corn gluten meal powder. Furthermore, in Japan, the free sulfite concentration of foods should be reduced to much less than 150 ppm.

It is an object of the present invention, which has been made in view of the above circumstances, to provide a corn wet milling product with a reduced sulfur dioxide concentration.

### Means for Solving the Problems

As a result of intensive studies regarding the object, the inventors have completed the present invention based on findings that the concentration of sulfur dioxide in corn wet milling products can be reduced by subjecting the corn wet milling products to filamentous fungal culture. More specifically, the present invention provides the following aspects.

<1> A product comprising a product resulting from exposing a corn wet milling product to a solid filamentous fungus culture.

<2> The product according to aspect <1>, wherein the corn wet milling product is one or more selected from the group consisting of corn gluten meal, corn gluten feed, and corn germ.

<3> The product according to aspect <1> or <2>, wherein the product has a sulfur dioxide content of 30 ppm or less.

<4> The product according to any one of aspects <1> to <3>, wherein the product has a sulfur dioxide content of 10 ppm or less.

<5> The product according to any one of aspects <1> to <4>, wherein the filamentous fungus is a non-toxin-producing fungus.

<6> The product according to aspect <5>, wherein the non-toxin-producing fungus is one or more selected from the group consisting of Aspergillus oryzae, Aspergillus sojae, Aspergillus luchuensis, Aspergillus niger, Aspergillus awamori, and Monascus pilosus.

<7> The product according to any one of aspects <1> to <6>, wherein the product contains an enzyme produced by the filamentous fungus.

<8> The product according to any one of aspects <1> to <7>, wherein the product contains a filamentous fungal cell polysaccharide.

<9> A solid filamentous fungus culture comprising a product produced by a method comprising: inoculating a corn wet milling product with a filamentous fungus; and subjecting the inoculated product to solid culturing.

<10> A composition comprising the product according to any one of aspects <1> to <9>.

<11> A food, animal feed, food additive, or animal feed additive comprising the product according to any one of aspects <1> to <9> or the composition according to aspect <10>.

<12> A method for producing a solid filamentous fungus culture, the method comprising:

inoculating a corn wet milling product with a filamentous fungus; and
subjecting, to solid culturing, the corn wet milling product inoculated with the filamentous fungus.

<13> The method according to aspect <12>, wherein the corn wet milling product contains sulfur dioxide.

### Effects of the Invention

The present invention provides a corn wet milling product with a reduced sulfur dioxide concentration.

### PREFERRED MODE FOR CARRYING OUT THE INVENTION

Hereinafter, embodiments of the present invention will be described. It should be noted that the embodiments are not intended to limit the present invention.

### 1. Solid Filamentous Fungus Culture

An embodiment of the present invention is directed to a solid filamentous fungus culture that is a culture product obtained by a process comprising: inoculating a corn wet milling product (a substrate) with a filamentous fungus; and subjecting the inoculated corn wet milling product to solid culturing. As used herein, the term “corn wet milling product” refers to a product produced when corn is subjected to a wet milling process. Examples of the corn wet milling product include, but are not limited to, corn gluten meal, corn gluten feed, and corn germ.

In particular, for ease of producing advantageous effects according to the present invention, the corn wet milling product is preferably corn gluten meal. Corn gluten meal is a material composed mainly of protein and starch separated from corn kernels by a wet milling process. Corn gluten meal is also called gluten meal.

On the other hand, the corn wet milling product may be free of corn starch since as mentioned above, sulfur dioxide can be easily removed by washing in the production of corn starch.

In the description, the corn wet milling product is also referred to as the “substrate”.

The solid culture may be performed using any known method. For example, the solid culture may be performed by a method comprising: inoculating the substrate with filamentous fungal seeds (spores); placing the inoculated substrate on a culture bed; and blowing, to the substrate, air with its temperature and humidity being controlled strictly.

The solid filamentous fungus culture typically has a sulfur dioxide content of 30 ppm or less, preferably less than 30 ppm, more preferably 20 ppm or less, even more preferably 10 ppm or less, furthermore preferably less than 10 ppm. The solid filamentous fungus culture according to an embodiment of the present invention having such a reduced sulfur dioxide content can be used as a food product.

The content of sulfur dioxide in the solid filamentous fungus culture may be measured by the colorimetric method (analytical method B) for measuring sulfur dioxide, defined in Appendix 3 of “Analytical Methods for Food Additives in Food, 2nd Edition”.

The filamentous fungus used to produce the solid filamentous fungus culture may be any species that is not harmful to animals that will ingest the culture and that is capable of utilizing the substrate for growth. Examples of such a filamentous fungus include Aspergillus and Monascus. In particular, for use as a food product, the filamentous fungus is preferably a non-toxin-producing fungus. For example, such a non-toxin-producing fungus is preferably a fungus that cannot express toxin production-related genes due to the accumulation of genetic factors, such as mutation, deletion, or transcriptional inhibition of genes related to the biosynthesis of toxin and has lost the ability to produce toxin. Specifically, such a non-toxin-producing fungus may be, for example, Aspergillus oryzae, Aspergillus sojae, Aspergillus luchuensis, Aspergillus niger, Aspergillus awamori, or Monascus pilosus. These filamentous fungus are commercially available in the form of spores for the production of fermented foods or available from National Institute of Technology and Evaluation Biological Resource Center (NBRC). The filamentous fungus may be a wild strain not modified genetically or a strain modified by genetic engineering, for example, to produce an increased level of an enzyme, which will be described later.

Examples of fungal toxins include aflatoxin, deoxynivalenol, ochratoxin, fumonisin, zearalenone, patulin, sterigmatocystin, and fusarium toxin.

The solid filamentous fungus culture may contain the filamentous fungus in one or both of living and dead states.

The solid filamentous fungus culture preferably contains an enzyme produced by the filamentous fungus. The enzyme produced by the filamentous fungus is typically, but not limited to, a degradative enzyme. Examples of the degradative enzyme include amylase, alkaline protease, acid protease, neutral protease, xylanase, β-glucanase, cellulase, tannase, phytase, lactase, lipase, pectinase such as polygalacturonase, xylanase-pectinase complex enzyme, and cellulase-protease-pectinase complex enzyme.

The solid filamentous fungus culture containing such an enzyme has an additional function. For example, the enzyme may be phytase, which catalyzes the chemical reaction to split off phosphoric acid from phytic acid. Phytic acid is considered to inhibit the absorption of minerals, such as calcium and zinc, in the animal body. Thus, the degradation of phytic acid by phytase can improve the absorption rate of minerals. Phosphorus resulting from the degradation of phytic acid can also be absorbed in the animal body.

For example, the enzyme may be cellulase or pectinase, which is a degradative enzyme that catalyzes the reaction to break down cellulose or pectin. Polysaccharides such as cellulose and pectin are among the components of plant cell walls. Various types of polysaccharides are known to be components of plant cell walls. They have various morphologies and complex compositions. In order to efficiently break down cell wall polysaccharides with complex structures, multiple degradative enzymes are preferably allowed to act stepwise. For example, multiple enzymes such as cellulase and pectinase may be allowed to act to break down cellulose and pectin. These enzymes can break down, with an improved efficiency, the cell walls of plant materials into digestible products.

For example, the enzyme may be tannase, which catalyzes the reaction to break down tannins. Some tannins strongly bind to proteins and other macromolecules to form complexes. Tannins form complex entanglements with plant cell wall components and may inhibit the degradation of cell walls. Tannase may be allowed to act to break down tannins. It can break down, with an improved efficiency, the cell walls of plant materials into digestible products.

By known genetic engineering methods, the filamentous fungus may be transformed so as to highly express the enzyme above. Some filamentous fungus are known to have a high level of expression of the amylase (AmyB) promoter or the enolase (enoA) promoter. Using known techniques, a chimeric gene may be formed by fusing, with the promoter, a gene encoding a desired one of the enzymes shown above and a terminator sequence corresponding to the promoter. Using known techniques, the chimeric gene may be introduced into the filamentous fungus. The resulting filamentous fungus will highly express the desired enzyme. The genome sequences of some filamentous fungus have already been analyzed and are publicly available in databases. Using such databases, primers may be designed based on the study of the sequences of high-expression promoters, terminators, and genes for encoding the desired enzyme. The desired gene sequence may be amplified by PCR using the designed primers and templates such as cDNA and genomic DNA and then used for transformation. The transformation process may include introducing the chimeric gene at a desired site in the genome using a known genome editing technique or may include introducing the chimeric gene into filamentous fungal cells to introduce the chimeric gene at any site in the genome. A known marker gene, such as niaD or ptrA, may be used for the selective culture of the transformed filamentous fungus.

The cloning of the gene to be highly expressed is preferably performed using template genomic DNA and cDNA derived from the same species as the filamentous fungus to be transformed. When genes derived from an organism belonging to the same species as the transformation target filamentous fungus are incorporated, no exogenous gene will be incorporated so that the safety of the filamentous fungus can be ensured. Such a cloning technique is called self-cloning.

The solid filamentous fungus culture preferably contains a filamentous fungal cell polysaccharide. The fungal cell is covered with a cell wall, which is composed mainly of polysaccharides. Examples of such polysaccharides include glucan, chitin, and chitosan. These polysaccharides are known to be involved in immunostimulatory activity. Thus, taking the solid filamentous fungus culture containing the polysaccharide is expected to be effective in strengthening the immune system. Proteins and lipids contained in the filamentous fugal cells will also be digested and absorbed as nutrients in the animal.

Corn gluten meal or some other by-products, which have a very high protein content and a low carbohydrate content, may allow only low efficiency growth of the filamentous fungus. Thus, a starchy material may be added to the corn wet milling product for the solid culture, and the solid filamentous fungus culture may contain such a starchy material. Examples of the starchy material include, but are not limited to, rice bran, bran, rice flour, and wheat flour. The starchy material may also be a gelatinized product produced through heating.

The types and proportions of enzymes produced by filamentous fungus are known to vary with substrate pH. Making substrate pH acidic (pH 2 to 4) can inhibit the growth of various microorganisms other than the desired filamentous fungus. Thus, a pH adjusting agent may be added to the corn wet milling product for the solid culture, and the solid filamentous fungus culture may contain such a pH adjusting agent. Examples of the pH adjusting agent include, but are not limited to, citric acid, calcium carbonate, sodium carbonate, phosphoric acid, succinic acid, tartaric acid, and lactic acid.

The solid filamentous fungus culture may be used as is or mixed with any additional material to form a composition. In the composition including the filamentous fungus culture, the additional component may be any suitable material, such as an additive or a common raw material used for food or feed. The additional component content may be adjusted as appropriate according to the desired effect. The composition may also contain any of various additives, such as antioxidants, pH adjusting agents, flavoring agents, various esters, organic acids, organic acid salts, inorganic acids, inorganic acid salts, inorganic salts, colorants, emulsifiers, preservatives, seasonings, and quality stabilizers.

The solid filamentous fungus culture or the composition may be fed to animals as is or may be added to other foods or animal feeds. The solid filamentous fungus culture or the composition may also be added to a food additive or an animal feed additive for use in other foods or animal feeds.

Examples of the target to which the solid filamentous fungus culture can be supplied include, but are not limited to, human, livestock animals, such as cows, pigs, goats, and poultry, crustaceans, such as shrimp and crab, and fish (including cultured fish). Examples of poultry includes chickens, canards, ducks, and geese.

As is clear from the above, embodiments of the present invention encompass the use of the solid filamentous fungus culture or composition according to an embodiment of the present invention for the production of a food, an animal feed, a food additive, or an animal feed additive. Embodiments of the present invention also encompass a method of producing a food, an animal feed, a food additive, or an animal feed additive, comprising the step of handling the solid filamentous fungus culture or composition according to an embodiment of the present invention. The step of handling the solid filamentous fungus culture or composition according to an embodiment of the present invention may comprise providing the solid filamentous fungus culture or composition according to an embodiment of the present invention or mixing the solid filamentous fungus culture or composition according to an embodiment of the present invention with an additional material to form a food, an animal feed, a food additive, or an animal feed additive.

### 2. Method for Producing the Solid Filamentous Fungus Culture

A method for producing the solid filamentous fungus culture is provided, comprising: inoculating the corn wet milling product with the filamentous fungus; and subjecting, to solid culturing, the corn wet milling product inoculated with the filamentous fungus. The method for producing the solid filamentous fungus culture may further comprise, before the step of inoculating with the filamentous fungus, a raw material processing step that includes adding water to the corn wet milling product, steaming the resulting mixture, and cooling the steamed mixture. The solid culturing of the filamentous fungus may be performed using any known method. For example, the production method may include a raw material processing step that includes adding water to the corn wet milling product, steaming the resulting mixture, and cooling the steamed mixture; a filamentous fungus inoculation step that includes inoculating, with the filamentous fungus, the corn wet milling product having undergone the raw material processing; and a solid culture step that includes subjecting, to solid culturing, the processed corn wet milling product inoculated with the filamentous fungus.

The corn wet milling product to be subjected to the solid culturing may contain sulfur dioxide. The concentration of sulfur dioxide in the corn wet milling product may be 100 ppm or more, 200 ppm or more, or 500 ppm or more. The solid filamentous fungus culture resulting from the process including the inoculation of the corn wet milling product with the filamentous fungus and the solid culturing has a reduced sulfur dioxide concentration of 30 ppm or less.

The raw material processing step may be performed using any known method for processing the corn wet milling product. Such a method may include, for example, adding water to the corn wet milling product; stirring the mixture; then steaming the mixture; and cooling the steamed mixture to a temperature that allows the substrate to be inoculated with the filamentous fungus. The term “corn wet milling product” is as defined above for the solid filamentous fungus culture.

The filamentous fungus inoculation step may be performed using any known method for inoculating the corn wet milling product with the filamentous fungus. After the raw material processing step, for example, the inoculation step may include dispersing seeds (spores) of the filamentous fungus onto the substrate. The filamentous fungus with which the corn wet milling product is to be inoculated may be any species, such as that shown above regarding the solid filamentous fungus culture.

The substrate may be steamed using any suitable method. For example, such a method may include adding, to the corn wet milling product, water in an amount of preferably 0.5 to 1.5 times, more preferably 0.7 to 1.3 times the amount of the corn wet milling product; stirring the mixture; and then steaming the mixture at a temperature of preferably 60 to 160° C., more preferably 90 to 130° C., for a time period of preferably 0.5 to 90 minutes, more preferably 2 to 60 minutes.

The steamed substrate may be inoculated with any suitable number of spores of the filamentous fungus. For example, the steamed substrate is preferably inoculated with 1.0×10³ to 1.0×10⁹, more preferably 1.0×10⁴ to 1.0×10⁸ of spores of the filamentous fungus per 1 g of the substrate.

The solid culture step may be performed using any suitable method for subjecting, to solid culturing, the corn wet milling product inoculated with the filamentous fungus. For example, such a method may include placing the inoculated substrate on a culture bed in a solid culture apparatus; and passing, through the spaces between the substrate particles, air with its temperature and humidity being controlled.

In the solid culture step, the temperature of the substrate changes over culturing time as the filamentous fungus grows and produces enzymes. Thus, during the solid culturing, at least one of the temperature, humidity, or flow rate of air being supplied to the substrate is preferably controlled to allow the culturing to proceed according to the desired substrate temperature course. This enables accurate control of the temperature and humidity during the culturing and allows the filamentous fungus to grow efficiently while suppressing the growth of other microorganisms.

It is also known that the types of enzymes produced by filamentous fungus vary with substrate temperature. Thus, the temperature of the substrate may be controlled so as to increase the production of the desired enzyme.

The temperature of the substrate is preferably, but not limited to, 10 to 55° C., more preferably 15 to 50° C. The temperature of the substrate during a period from the start of culturing to when a certain period has passed may be different from that after the certain period. For example, a method may be used comprising: controlling the temperature of the substrate to 30° C. during a period from the start of culturing to when 24 hour culturing is completed; and controlling the temperature of the substrate to 25° C. after the 24 hour culturing.

While the air may have any suitable humidity, it preferably has a relative humidity of 50 to 99%, more preferably 70 to 99%.

The water content of the substrate is preferably controlled during the production of the solid filamentous fungus culture. The water content may be controlled by water sprinkling, drying, or any other method. Water sprinkling or drying may be performed during the solid culture step or after the completion of the solid culture step. A known method for increasing solid culturing efficiency includes sprinkling water to control the water content of the substrate during the solid culture step. Sprinkling water during the solid culture step can maintain the water activity at a level suitable for the growth of the filamentous fungus and provide more active growth of the filamentous fungus and more active enzyme production. To enhance the storage stability of the solid filamentous fungus culture, the solid culture step may be followed by drying to control the water content of the substrate. This prevents the deterioration of the solid filamentous fungus culture and other materials mixed therewith and allows quality stabilization and long-term storage.

In the solid culture step, the culturing may be performed for any suitable period of time. For example, the culturing may be performed until the substrate surface is covered with the mycelia of the filamentous fungus. Specifically, the culturing may be performed for a time period of 30 to 100 hours or 40 to 90 hours.

As mentioned above, the corn wet milling product, which sometimes has a very high protein content and a low carbohydrate content, may allow only low efficiency growth of the filamentous fungus. Thus, a starchy material may be added to the wet milling product, and the resulting mixture may be subjected to the solid culturing. Examples of the starchy material that may be added to the corn wet milling product are those listed above for the solid filamentous fungus culture. The starchy material may be added to the corn wet milling product during the raw material processing step, during the filamentous fungus inoculation step, during the solid culture step, or during all of these steps.

As mentioned above, the types and proportions of enzymes produced by the filamentous fungus depend on the pH of the substrate. Making the pH of the substrate acidic (pH 2 to 4) can inhibit the growth of various microorganisms other than the desired filamentous fungus. Thus, a pH adjusting agent may be added to the corn wet milling product for the solid culturing. A known method, such as addition of a pH adjusting agent, may be used to control the pH. Examples of the pH adjusting agent are those listed above for the solid filamentous fungus culture. The pH adjustment may be performed during the raw material processing step, during the filamentous fungus inoculation step, during the solid culture step, or during all of these steps.

### EXAMPLES

Hereinafter, the present invention will be specifically described with reference to examples, which are not intended to limit the present invention.

### Example 1

In Example 1, corn gluten meal (a corn wet milling product) was used as a raw material, inoculated with Aspergillus oryzae, and then subjected to solid culturing.

To 60 kg of corn gluten meal with a water content of 10.8% was added 67.4 L of water. The resulting mixture was stirred and then steamed at 100° C. for 30 minutes. The steamed corn gluten meal was cooled to around 35° C., then inoculated with Aspergillus oryzae (Three Dia (additive-free), Higuchi Matsunosuke Shoten Co., Ltd.), which is commercially available as a seed fungus for use in soy source production, and then subjected to mixing for uniform inoculation. The steamed corn gluten meal was inoculated with approximately 2×10⁶ of the spores per 1 g of the corn gluten meal. The seed fungus Aspergillus oryzae is a non-toxin-producing fungus. The substrate inoculated with the seed fungus was placed on the culture bed in a solid culture apparatus. After the placement, the substrate was leveled to a uniform thickness and then started to be subjected to solid culturing. During the culturing, the temperature of the substrate was strictly controlled by supplying, from below the culture bed, air with its temperature and humidity being controlled and blowing the air to the substrate placed on the culture bed. The temperature and humidity of the air being supplied were controlled such that the temperature of the substrate was maintained at 30°° C. during a period from the start of culturing to when the culturing was performed for 24 hours and then maintained at 25°° C. after the 24 hour culturing. The relative humidity of the air being supplied was controlled to fall within the range of 90 to 99% constantly. During the culturing, the substrate was appropriately stirred with a stirrer attached to the solid culture apparatus to ensure uniform culturing conditions. After the culturing was performed for 45 hours, it was confirmed that the surface of the corn gluten meal granules (the substrate) was sufficiently covered with the mycelia of the filamentous fungus. The substrate was further dried overnight by air blowing for the purpose of controlling the water content of the substrate, giving a solid filamentous fungus culture.

The sulfur dioxide content of the corn gluten meal before the culturing and that of the solid filamentous fungus culture were measured by the colorimetric method (analytical method B) for measuring sulfur dioxide, defined in Appendix 3 of “Analytical Methods for Food Additives in Food, 2nd Edition”. While the corn gluten meal had a sulfur dioxide content of 380 ppm before the culturing, the solid filamentous fungus culture had a sulfur dioxide content of 21 ppm.

### Example 2

In Example 2, corn gluten meal (a corn wet milling product) was used as a raw material, inoculated with Monascus pilosus, and then subjected to solid culture.

To 15 kg of corn gluten meal with a water content of 10.6% was added 11.8 kg of water. The resulting mixture was stirred and then steamed under pressure at 121°° C. for 3 minutes. The steamed corn gluten meal was cooled to around 35° C., then inoculated with Monascus pilosus (NBRC4520) obtained from NBRC, and then subjected to mixing for uniform inoculation. The steamed corn gluten meal was inoculated with approximately 2×10⁶ of the spores per 1 g of the corn gluten meal. The seed fungus Monascus pilosus is a non-toxin-producing fungus. The substrate inoculated with the seed fungus was placed on the culture bed in a solid culture apparatus. After the placement, the substrate was leveled to a uniform thickness and then started to be subjected to solid culturing. During the culturing, the temperature of the substrate was strictly controlled by supplying, from below the culture bed, air with its temperature and humidity being controlled and blowing the air to the substrate placed on the culture bed. The temperature and humidity of the air being supplied were controlled such that the temperature of the substrate was maintained at 30° C. from the start to end of culturing. The relative humidity of the air being supplied was controlled to fall within the range of 90 to 99% constantly. During the culturing, the substrate was appropriately stirred to ensure uniform culturing conditions. After the culturing was performed for 89 hours, it was confirmed that the surface of the corn gluten meal granules (the substrate) was sufficiently covered with the mycelia of the filamentous fungus. As a result, a solid filamentous fungus culture was obtained. The substrate was further dried overnight by air blowing for the purpose of controlling the water content of the substrate, giving a solid filamentous fungus culture.

The sulfur dioxide content of the corn gluten meal before the culturing and that of the solid filamentous fungus culture were measured by the same method as in Example 1. While the corn gluten meal had a sulfur dioxide content of 340 ppm before the culturing, the solid filamentous fungus culture had a sulfur dioxide content of 14 ppm.

1. A product comprising a product resulting from exposing a corn wet milling product to a solid filamentous fungus culture.

2. The product according to claim 1, wherein the corn wet milling product is one or more selected from the group consisting of com gluten meal, corn gluten feed, and corn germ.

3. The product according to claim 1, wherein the product has a sulfur dioxide content of 30 ppm or less.

4. The product according to claim 1, wherein the product has a sulfur dioxide content of 10 ppm or less.

5. The product according to claim 1, wherein the filamentous fungus is a non-toxin-producing fungus.

6. The product according to claim 5, wherein the non-toxin-producing fungus is one or more selected from the group consisting of Aspergillus oryzae, Aspergillus sojae, Aspergillus luchuensis, Aspergillus niger, Aspergillus awamori, and Monascus pilosus.

7. The product according to claim 1, wherein the product contains an enzyme produced by the filamentous fungus.

8. The product according to claim 1, wherein the product contains a filamentous fungal cell polysaccharide.

9. A solid filamentous fungus culture comprising a product produced by a method comprising: inoculating a corn wet milling product with a filamentous fungus;
and subjecting the inoculated product to solid culturing.

10. A composition comprising the product according to claim 1.

11. A food, animal feed, food additive, or animal feed additive comprising the product according to claim 1.

12. A method for producing a solid filamentous fungus culture, the method comprising:
inoculating a corn wet milling product with a filamentous fungus; and
subjecting, to solid culturing, the corn wet milling product inoculated with the filamentous fungus.

13. The method according to claim 12, wherein the corn wet milling product contains sulfur dioxide.

14. A food, animal feed, food additive, or animal feed additive comprising the composition according to claim 10.
